(19) United States
(12) Patent Application Publication
(10) Pub. No.: US 2026/0108453 A1
(43) Pub. Date: Apr. 23, 2026

(54) REMINERALIZING PROBIOTIC CHEWABLE TABLET

(71) Applicant: Angelika Batres Navarro, Coral Gables, FL (US)

(72) Inventor: Angelika Batres Navarro, Coral Gables, FL (US)

(21) Appl. No.: 18/918,320

(22) Filed: Oct. 17, 2024

(51) Int. Cl. A61K 8/99; A61K 8/19; A61K 8/60; A61Q 11/00

(52) U.S. Cl. CPC A61K 8/99; A61K 8/19; A61K 8/60; A61Q 11/00

(57) ABSTRACT

A chewable tablet a first probiotic strain of bacteria that inhibits Streptococcus mutans; a second probiotic strain of bacteria that inhibits one or more of Porphyromonas gingivalis, Treponema denticola, and Tannerella forsythia (i.e., the red complex); a remineralizing agent such as hydroxyapatite with a micron-level particle size; and a sugar alcohol such as xylitol.

### BACKGROUND OF THE INVENTION

The present invention relates to the treatment and prevention of dental caries and, more particularly, to a remineralizing probiotic chewable tablet.

In the United States, dental problems are the second most commonly occurring medical condition, trailing only the common cold. Dental caries (tooth decay and cavities) and periodontal disease are the two most substantial oral health problems in the United States and are among the most common preventable chronic diseases. In fact, dental caries is the most common preventable chronic disease in children.

Microscopic defects are caused in tooth enamel due to changes in pH after eating sweets or when cavity-causing bacteria overpopulate the teeth surfaces, causing decay. Streptococcus mutans (S. mutans) is the most prominent caries-causing bacteria. A group of three oral bacteria referred to as the “red complex” is responsible for gingivitis and periodontitis, including Porphyromonas gingivalis, Treponema denticola, and Tannerella forsythia (formerly referred to as Bacteroides forsythus).

Commercially available probiotics may have one or two strains of healthy bacteria in vegetable-based capsule formulations. The capsules cannot be chewed to keep the contents in contact with the subject's teeth long enough to have any significant caries-preventing results. Some commercially available toothpastes contain hydroxyapatite. No currently available, orally applied product contains multiple probiotic strains, a sugar alcohol to target caries-causing bacteria, and remineralizing agents.

As can be seen, there is a need for a chewable tablet that can prevent bacteria growth and can be part of an easy routine that decreases dental caries.

### SUMMARY OF THE INVENTION

In one aspect of the present invention, a chewable tablet, comprising a first probiotic strain of bacteria effective to inhibit Streptococcus mutans; a second probiotic strain of bacteria effective to inhibit one or more of Porphyromonas gingivalis, Treponema denticola, and Tannerella forsythia (i.e., the red complex); a remineralizing agent; and a sugar alcohol.

The tablet includes ingredients that prevent decay, freshen breath, improve oral and gut health, and remineralize enamel. The chewable composition is safe to consume and is pleasant to use.

These and other features, aspects and advantages of the present invention will become better understood with reference to the following drawings, description, and claims.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a chewable tablet comprising probiotic bacteria with a remineralizing agent and a sugar alcohol. Without being bound by theory, the ingredients are believed to have a synergistic effect because each ingredient performs a distinct function that impacts the processes affected by each other ingredient.

The composition disclosed herein contains billions of colony-forming units (CFU) of 6 strains of bacteria and leads to a shift in oral bacteria from cavity causing bacteria to healthy bacteria. The oral flora is repopulated with healthy, non-disease-causing bacteria.

The composition may include about 1-4 bacteria strains that act mainly against S. mutans and about 5-6 bacteria strains that target the red complex. Suitable probiotic strains may include but are not limited to any combination selected from the group consisting of Akkermansia muciniphila, Bacillus subtilis, Bifidobacterium animalis, Bifidobacterium bifidum, Bifdobacterium longum, Bifidobacterium breve, Bifidobacterium lactis, Lactobacillus acidophilus, Lactobacillus casei, Lactobacillus LAFTI, Lactobacillus plantarum, Lactobacillus reuteri, Lactobacillus rhamnosus, Lactobacillus paracasei, Lactobacillus bulgaricus, Lactobacillus gasseri, Lactobacillus fermentum, Lactobacillus brevis, Lactobacillus cellobiosus, Lactobacillus salivarius, Streptococcus oligofermentans, Streptococcus sanguinis, Streptococcus thermophilus, and Lactococcus lactis. For example, Lactobacillus casei targets the gums, inhibits S. mutans (dental caries), inhibits S. sobrinus (dental caries), reduces C-reactive protein, reduces Irritable Bowel Syndrome, and reduces inflammation. Lactobacillus paracasei targets periodontal disease and binds to Porphyromonas gingivalis (gum disease). Lactobacillus salivarius targets the gums, inhibits Tannerella forsythia (gum disease), inhibits Porphyromonas gingivalis, works in tandem with Lactobacillus plantarum, inhibits S. mutans, reduces caries, reduces strep throat, inhibits Strep pyogenes (strep throat), reduces ulcerative colitis, inhibits Candida, and inhibits Salmonella. Lactobacillus plantarum targets periodontal disease, works in tandem with L. salivarius, reduces Irritable Bowel Syndrome, and reduces Interleukin 6 levels. Lactobacillus rhamnosus targets periodontal disease, reduces Candida, reduces caries, inhibits S. mutans, inhibits S. sobrinus, and inhibits inflammation. Preferably, the bacteria are proven to contribute to a healthy gut microbiome as well.

In some embodiments, the composition may include a strain of bacteria that decreases sulfur in the mouth, thereby helping to remediate bad breath.

The remineralizing agent has a micron-level particle size that targets enamel directly, filling gaps or voids in the enamel. The micron size helps remineralize defects on enamel and minimally on dentin. For example, micron-level microcrystalline hydroxyapatite is a biocompatible and bioavailable remineralizing agent that is naturally the main component of teeth and bones. The remineralizing agent may be present in any suitable amount, such as about 0.05% to about 50% by weight.

Nano-scale hydroxyapatite, e.g., having a particle size of from 1 to 100 nm, is prohibited for use in cosmetics in Europe. They have been shown to cross the membrane barrier and damage the nephrons in the kidneys. As such, nano-scale hydroxyapatite may be excluded from the composition.

The sugar alcohol kills bacteria that cause cavities. The sugar alcohol may be selected from the group consisting of arabitol, erythritol, glycerol, mannitol, sorbitol, xylitol, lactitol, isomalt, maltitol and hydrogenated starch hydrolysates (HSH), and any combination thereof. Xylitol, for example, is toxic to S. mutans, disrupting the metabolism of the bacteria, thereby reducing the S. mutans population in the oral cavity. It also helps prevent plaque buildup on tooth surfaces. The sugar alcohol may be present in any suitable amount, such as about 10 mg to about 50 mg per tablet.

In some embodiments, a weight ratio of xylitol to micron-sized hydroxyapatite may be about 10:1 to about 1:10.

In some embodiments, the chewable tablet comprises a flavoring agent, which may be selected from but is not limited to: spearmint, cranberry extract, blueberry extract, and combinations thereof.

The chewable tablet may include any suitable excipient base formula known in the art.

To use the tablets described herein, the user may take 1-2 chewable tablets after brushing every night, keep the chewed tablet(s) in the mouth for 30 seconds, and then swallow or spit out the remainder. Hydroxyapatite microcrystals lodge on the user's teeth and generally remain on the surface of the molars for at least 4 minutes.

In a non-limiting example, a chewable tablet may be formed from a blended oral probiotic composition, comprising:
Lactobacillus paracasei 2 cfu
Lactobacillus plantarum 1 cfu
Lactobacillus rhamnosus 1 cfu
Lactobacillus casei 1 cfu
Lactobacillus salivarius 1 cfu
30 mg xylitol
110 mg 840-micron hydroxyapatite
Spearmint flavor

It should be understood, of course, that the foregoing relates to exemplary embodiments of the invention and that modifications may be made without departing from the spirit and scope of the invention as set forth in the following claims.

What is claimed is:

1. A chewable tablet, comprising:
a first probiotic strain of bacteria effective to inhibit Streptococcus mutans;
a second probiotic strain of bacteria effective to inhibit one or more of Porphyromonas gingivalis, Treponema denticola, and Tannerella forsythia;
a remineralizing agent; and
a sugar alcohol.

2. The chewable tablet of claim 1, wherein the first probiotic strain is selected from the group consisting of Akkermansia muciniphila, Bacillus subtilis, Bifidobacterium animalis, Bifidobacterium bifidum, Bifdobacterium longum, Bifidobacterium breve, Bifidobacterium lactis, Lactobacillus acidophilus, Lactobacillus casei, Lactobacillus LAFTI, Lactobacillus plantarum, Lactobacillus reuteri, Lactobacillus rhamnosus, Lactobacillus paracasei, Lactobacillus bulgaricus, Lactobacillus gasseri, Lactobacillus fermentum, Lactobacillus brevis, Lactobacillus cellobiosus, Lactobacillus salivarius, Streptococcus oligofermentans, Streptococcus sanguinis, Streptococcus thermophilus, Lactococcus lactis, and any combination thereof.

3. The chewable tablet of claim 1, wherein the second probiotic strain is selected from the group consisting of Akkermansia muciniphila, Bacillus subtilis, Bifidobacterium animalis, Bifidobacterium bifidum, Bifdobacterium longum, Bifidobacterium breve, Bifidobacterium lactis, Lactobacillus acidophilus, Lactobacillus casei, Lactobacillus LAFTI, Lactobacillus plantarum, Lactobacillus reuteri, Lactobacillus rhamnosus, Lactobacillus paracasei, Lactobacillus bulgaricus, Lactobacillus gasseri, Lactobacillus fermentum, Lactobacillus brevis, Lactobacillus cellobiosus, Lactobacillus salivarius, Streptococcus oligofermentans, Streptococcus sanguinis, Streptococcus thermophilus, Lactococcus lactis, and any combination thereof.

4. The chewable tablet of claim 1, wherein the sugar alcohol is selected from the group consisting of arabitol, erythritol, glycerol, mannitol, sorbitol, xylitol, lactitol, isomalt, maltitol and hydrogenated starch hydrolysates (HSH), and any combination thereof.

5. The chewable tablet of claim 1, wherein the remineralizing agent is hydroxyapatite.

6. The chewable tablet of claim 1, wherein the remineralizing agent has a mean particle size of about 1 μm to about 100 μm.

7. The chewable tablet of claim 1, wherein each of the first probiotic strain and the second probiotic strain are present in a colony-forming unit ratio of about 1:1 to about 2:1.

8. The chewable tablet of claim 1, further comprising an excipient base and/or a flavoring agent.

9. The chewable tablet of claim 1, wherein the first probiotic strain is selected from the group consisting of Lactobacillus casei, Lactobacillus salivarius, Lactobacillus rhamnosus, Lactobacillus plantarum, and any combination thereof; the second probiotic strain is selected from the group consisting of Lactobacillus casei, Lactobacillus paracasei, Lactobacillus rhamnosus, Lactobacillus salivarius, and any combination thereof; the remineralizing agent is hydroxyapatite with a mean particle size of about 1 μm to about 10 μm; and the sugar alcohol is xylitol.
